(19) United States
(12) Patent Application Publication
(10) Pub. No.: US 2017/0350876 A1
(43) Pub. Date: Dec. 7, 2017

(54) BIOSENSOR FOR SINGLE CELL ANALYSIS

(71) Applicants: Mohammad Abdolahad, Tehran (IR); Milad Gharooni, Tehran (IR); Alireza Alikhani, Shiraz (IR); Hamed Abiri, Tehran (IR)

(72) Inventors: Mohammad Abdolahad, Tehran (IR); Milad Gharooni, Tehran (IR); Alireza Alikhani, Shiraz (IR); Hamed Abiri, Tehran (IR)

(21) Appl. No.: 15/681,373

(22) Filed: Aug. 19, 2017

Related U.S. Application Data
(60) Provisional application No. 62/376,919, filed on Aug. 19, 2016.

(51) Int. Cl. G01N 33/483; C23C 14/06; G01N 15/10; G01N 27/04; C23C 14/34; G01N 15/00

(52) U.S. Cl. CPC G01N 33/4836; G01N 27/04; C23C 14/34; C23C 14/06; G01N 15/1031; G01N 2015/0065; G01N 2015/1006

(57) ABSTRACT

A biosensor for single cell analysis is disclosed. The biosensor includes a substrate, an array of electrodes, and a passivation layer. The substrate includes a roughened surface, where the array of electrodes is patterned on the roughened surface. Each electrode includes a distal tip and a proximal end. The passivation layer is deposited on top of the biosensor and includes a microwell around the distal tip of an electrode. A single cell is trapped within the microwell and adhered onto the distal tip of the electrode for further single cell analysis.

### CROSS-REFERENCE TO RELATED APPLICATION

This application claims the benefit of priority from pending U.S. Provisional Patent Application Ser. No. 62/376,919, filed on Aug. 19, 2016, and entitled “BIOELECTRICAL IMPEDIMETRIC SENSOR FOR SINGLE CELL ANALYSIS AND APPLICATIONS THEREOF IN CANCER THERAPEUTIC PURPOSES,” which is incorporated herein by reference in its entirety.

### TECHNICAL FIELD

The present disclosure generally relates to devices for single cell analysis, and particularly, to a biosensor for single cell analysis, such as extreme drug resistance (EDR) assays. The method further relates to a method for fabricating a biosensor for single cell analysis.

### BACKGROUND

Heterogeneity of cellular metabolism during disease transformation or therapeutic treatments has resulted in increased understanding of physiological functions of single cells. Apart from scientific benefits to achieve new biological phenomena in cell study, many diagnostic and therapeutic protocols in non-communicable diseases were introduced by single cell analysis and tracing pathological evidences through a single cell. However, an outstanding challenge for many modern single-cell assays is damaging the cells during the analysis and limitation in time dependent monitoring of a treated cell. For instance, the nature of biochemical analysis causes cellular damaging and lysis.

2

Moreover, non-invasive methods to maintain the investigated cell for time dependent monitoring has been widely studied because of its importance in some crucial cases such as drug resistance in cancer. However, some of the non-invasive procedures such as Raman spectroscopy, or optical bio sensing involve complicated operating procedures. In addition, they present insufficient data on biological state of the cell.

Bioelectrical monitoring is one of the non-invasive methods. Although the procedures reported based on electrical probing might not induce cell disruption, the indirect connection between recording electrodes and cell membrane, which is mostly used in microfluidic approaches reduces the quality of response and limits the precision of the results. A limitation associated with some of the biosensors is the fact that they are based on microfluidic approach in which the cell is held in a solution by micro cavity without direct access of the electrodes to its membrane. Locating the cells in desired place between electrodes, for example, by a micropipette or using a voltage phase controlled method is some experimentally complicated procedure which reduces the applications of such devices. However, precise location of the cell between the recording electrodes are crucial to suppress electrical noises in the response of such devices.

Hence, there is a need for an electrical biosensor for single cell analysis in order to overcome the shortcomings associated with conventional electrical biosensors, particularly providing a direct non-invasive contact with a single cell membrane. In addition, there is a need for an electrical biosensor, in which a single cell may be randomly trapped near the sensing electrodes easily and without procedure complications. Moreover, an electrical biosensor for single cell analysis with a structure that provides more intensified electrical field and electrical signals in order to obtain results with more precision and accuracy.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a biosensor for single cell analysis. The biosensor may include a substrate, an array of electrodes, and a passivation layer. The substrate may include a roughened surface, where the array of electrodes may be patterned on the roughened surface. Each electrode may include a distal tip and a proximal end. The passivation layer may be deposited on top of the biosensor and may include a microwell around the distal tip of an electrode. A single cell may be trapped within the microwell and adhered onto the distal tip of the electrode for further single cell analysis.

In one implementation, the single cell may be trapped within the microwell by dropwise inserting a solution of a population of cells, where the solution may include the single cell.

In an exemplary embodiment, the roughened surface may include a nano-roughened surface including nanosized features with diameter of less than about 100 nm and height of less than about 200 nm. In another exemplary embodiment, the substrate may include a quartz substrate, or a glass substrate, or a silicone-based substrate, or a Pyrex substrate, or a Poly(methyl methacrylate) (PMMA) substrate, or combinations thereof.

In some exemplary implementations, the microwell may include a circular cavity with a diameter between about 20 μm and about 50 μm and a depth between about 2 μm and about 5 μm. In an exemplary embodiment, the array of electrodes may include an array of Gold (Au) microelectrodes with a thickness of less than about 20 nm. In another exemplary embodiment, the array of electrodes may include a Titanium/Gold (Ti/Au) bilayer with a thickness of less than about 20 nm.

In some exemplary implementations, the biosensor may further include a layer of Titanium (Ti) between the array of electrodes and the roughened surface, which may be configured to improve the adhesion of the array of electrodes onto the roughened surface.

In an exemplary embodiment, the passivation layer may include an electrically insulator layer that may be formed by spinning a photoresist layer on an area on top of the biosensor except the distal tip and the proximal end of the array of electrodes. The passivation layer may include an overbaked photoresist polymeric layer with a thickness of less than about 5 μm.

In some exemplary implementations, the biosensor may be placed along or be a part of an electrical measurement system and the electrical measurement system may include the biosensor, an electrical signal extraction board, and a processor. The electrical signal extraction board may be configured to apply a voltage on the array of electrodes and to receive an electrical signal from the array of electrodes and the processor may be configured to store and analyze the electrical signal received by the electrical signal extraction board. The proximal ends of the array of electrodes of the biosensor may be electrically connected to the electrical signal extraction board and the electrical signal extraction board may be electrically connected to the processor. In an example, the electrical signal may include electrical impedance. In some examples, the applied voltage may be 40 mV and the voltage may be applied with a frequency between about 1 KHz and about 150 KHz.

In an exemplary embodiment consistent with principles of the present disclosure, a method for fabricating a biosensor for single cell analysis is disclosed. The method may include cleaning a substrate, roughening a surface of the clean substrate to form a roughened surface on the clean substrate, depositing a Titanium/Gold (Ti/Au) bilayer on the roughened surface, patterning the Ti/Au bilayer to form an array of electrodes, spinning a passivation layer on the roughened surface and the array of electrodes, and patterning the passivation layer to form a microwell. The Ti/Au bilayer may include a Ti layer deposited on the roughened surface and an Au layer deposited on the Ti layer. Moreover, the microwell may include a cavity around a distal tip of an electrode of the array of electrodes and the microwell may form a trap for capturing a single cell and adhering the single cell on the distal tip of the electrode.

In an implementation, roughening the surface of the clean substrate may include holding the clean substrate in a reactive ion etching (RIE) system. In another implementation, roughening the surface of the clean substrate may include placing the clean substrate in a buffer HF (Hydrogen fluoride) solution for less than 30 min.

In an exemplary embodiment, the bilayer of Ti/Au may be deposited on the roughened surface using a sputtering process. In another exemplary embodiment, patterning the Ti/Au bilayer may include patterning the Ti/Au bilayer using a lithography process. In an example, spinning a passivation layer may include depositing a photoresist layer on the roughened surface and the array of electrodes. Furthermore, patterning the passivation layer may include patterning the passivation layer using a lithography process.

In some implementations, the method may further include overbaking the biosensor at a temperature of more than about 100° C. to remove toxic materials from the passivation layer.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a schematic view of an implementation of a biosensor for single cell analysis, consistent with one or more exemplary embodiments of the present disclosure.

FIG. 1B illustrates a schematic view of an implementation of an electrical measurement system, consistent with one or more exemplary embodiments of the present disclosure.

FIG. 2 illustrates a method for fabricating a biosensor for single cell analysis, consistent with one or more exemplary embodiments of the present disclosure.

FIG. 3A illustrates an implementation of a clean substrate, consistent with one or more exemplary embodiments of the present disclosure.

FIG. 3B illustrates an implementation of a clean substrate with a roughened surface, consistent with one or more exemplary embodiments of the present disclosure.

FIG. 3C illustrates an implementation of a clean substrate with a roughened surface and a bilayer of Titanium/Gold (Ti/Au) deposited on the roughened surface, consistent with one or more exemplary embodiments of the present disclosure.

FIG. 3D illustrates an implementation of an array of electrodes patterned on a roughened surface of a biosensor, consistent with one or more exemplary embodiments of the present disclosure.

FIG. 3E illustrates an implementation of a passivation layer spun on the surface of a biosensor, consistent with one or more exemplary embodiments of the present disclosure.

FIG. 3F illustrates an implementation of a biosensor with a patterned passivation layer with microwells around distal tip of the array of electrodes, consistent with one or more exemplary embodiments of the present disclosure.

FIG. 4A illustrates a three dimensional (3D) atomic-force microscopy (AFM) image of a nano-roughened surface of a substrate, consistent with one or more exemplary embodiments of the present disclosure.

FIG. 4B illustrates a line atomic-force microscopy (AFM) image of a nano-roughened surface of a substrate, consistent with one or more exemplary embodiments of the present disclosure.

FIG. 5 illustrates an optical image of the patterned array of electrodes (left) and an optical image of a magnified portion of the patterned array of electrodes (right), consistent with one or more exemplary embodiments of the present disclosure.

FIG. 6 illustrates an optical image of the array of electrodes with patterned passivation layer including microwells (left) and an optical image of a magnified portion of the array of electrodes with patterned passivation layer (right) before cell attachment, consistent with one or more exemplary embodiments of the present disclosure.

FIG. 7 illustrates an optical image of an individual MCF-7 single cell trapped within a microwell and attached on the electrodes tip after dropping the cells solution (t=0) (left) and an optical image of a magnified portion after cell attachment (time=2 hours) (right), consistent with one or more exemplary embodiments of the present disclosure.

FIG. 8 illustrates results of a MTT assay for MCF-7 cells cultured on a bare quartz and a quartz coated by an overbaked photoresist layer, consistent with one or more exemplary embodiments of the present disclosure.

FIG. 9 illustrates optical microscopy images from a bare quartz surface and a quartz patterned surface by photoresist passivation layer, consistent with one or more exemplary embodiments of the present disclosure.

FIG. 10A illustrates impedance value of non-treated single MCF-7 cells attached between the couple electrodes (about 4 hours after dropping the cell, T=0), consistent with one or more exemplary embodiments of the present disclosure.

FIG. 10B illustrates impedance changes of a non-treated cell (CTRL) during time evolution, consistent with one or more exemplary embodiments of the present disclosure.

FIG. 10C illustrates impedance changes of a treated cell after treating the cell by MBZ with a low concentration of about 2.1 nM during time, consistent with one or more exemplary embodiments of the present disclosure.

FIG. 10D illustrates impedance changes of a treated cell after treating the cell by MBZ with a high concentration of about 10.5 nM during time, consistent with one or more exemplary embodiments of the present disclosure.

FIG. 10E illustrates impedance changes of a treated cell after treating the cell by PTX with a low concentration of about 0.1 nM during time, consistent with one or more exemplary embodiments of the present disclosure.

FIG. 10F illustrates impedance changes of a treated cell after treating the cell by PTX with a high concentration of about 1 nM during time, consistent with one or more exemplary embodiments of the present disclosure.

FIG. 11A illustrates confocal images of CTRL single cell at T=20 hours after drug treatment, consistent with one or more exemplary embodiments of the present disclosure.

FIG. 11B illustrates confocal images of MBZ treated single cell at T=20 hours after drug treatment, consistent with one or more exemplary embodiments of the present disclosure.

FIG. 11C illustrates confocal images of PTX treated single cell at T=20 hours after drug treatment, consistent with one or more exemplary embodiments of the present disclosure.

FIG. 12A illustrates average changes of impedance (derived from mean value in all of swap frequencies) for single MCF-7 cells treated by different doses of PTX and MBZ drugs analyzed by a biosensor with a nano-roughened surface in the frequency of about 4 kHz, consistent with one or more exemplary embodiments of the present disclosure.

FIG. 12B illustrates average changes of impedance (derived from mean value in all of swap frequencies) for single MCF-7 cells treated by different doses of PTX and MBZ drugs analyzed by a biosensor with a non-nano-roughened (smooth) surface in the frequency of about 4 kHz, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

In an active and complex electronic system, cells exhibit distinguishable electrical behavior during various physiological and pathological states. Moreover, many crucial changes in cell metabolism are highly correlated with electrochemical exchanges between a cell's inner and outer parts or bioelectrical changes in cytoskeleton, cytoplasm, nucleus, and membrane. The ability to translate a cell's physiological or pathological states to electrical patterns would be of great value for conducting a live analysis of a single cell. Many modern devices and methods have been introduced for electrical measurement of single cells. However, these electrical devices, particularly electrical biosensors involve some drawbacks and limitations such as the devices and the corresponding methods being too complicated, indirect contact between the single cell and electrodes that reduces the accuracy of the analysis results, etc. Moreover, some of these devices are based on the electrical driving force to fix the location of the cells, which may induce alteration in the cells due to applying an electrical field in the first step. Moreover, some approaches entail utilizing an inverted microcontact printing method on topographically structured polystyrene chips for arrayed micro-3-D culturing of single cells, where the fabrication process may be very complicated, i.e., about seven sequential steps either in constructing the cell adhesion layer or forming electrodes for signal extraction. In addition, many chemical processes may be used in the fabrication procedure.

Herein, in order to overcome shortcomings related to the conventional biosensors for single cell analysis, an electrical biosensor is described for noninvasive single cell analysis with high accuracy that may be utilized with less complications so that a single cell may be captured or trapped in a direct contact with sensing electrodes through a simple way. Furthermore, a method for fabrication a biosensor for single cell analysis is disclosed herein. Simple fabrication process, easy usage, and direct and accurate signal extraction are some of the main advantages of the present disclosure.

In this regard, the biosensor disclosed herein may be used for single cell assessments of a cell's behavior changes, for example, during therapeutic treatments such as drug incubation or ray therapy which would biologically induce one of the cell's sub-organelles, might also affect bioelectrical response of an exposed cell. For instance, anti-tubulin drugs may be considered to be one of the most important groups of anti-cancer drugs. The anti-tubulin drugs may cause the obstruction of microtubules (MTs) polymerization (lengthening) or depolymerization (shortening), which highly affects the dynamics of the spindles and results in the mitotic arrest and cell death. Microtubules are involved in a diverse range of cellular functions including mitosis and meiosis, motility, maintenance of cell shape and intracellular trafficking of macromolecules and organelles. Functional disturbances induced in the drug treated cancer cells may be diagnosed by membrane monitoring as the crucial organelles in cells vital cycle. A diverse range of structurally dissimilar compounds may interact with the tubulin/microtubule system and function as antimitotic agents. These antimitotic agents may be divided into two major classes: those that bind preferentially to α/β-tubulin heterodimers and inhibit polymer assembly, and those with a binding site on the polymer that stabilizes microtubules. Paclitaxel is one of several cytoskeletal drugs that target tubulin and Mebendazole (Mbz) (methyl5-benzoyl-2-benzimidazole-carbamate) is a prototype benzimidazole carbamate that was initially used for the treatment of intestinal roundworms infections that is applied in treating many types of cancers such as lung cancer, gastric cancer and colon cancer were also reported. Accordingly, the biosensor disclosed herein may be utilized for monitoring the effect of anticancer drugs on single breast cancer cells.

According to some exemplary implementations, the biosensor may include an array of gold electrodes patterned on a nano-roughened surface of a substrate that may provide direct contacts between sharp tips of the electrodes and cell membrane. The whole of the surface of the biosensor except a zone that may include a microcircle surrounding a sensing region may be passivated by an overbaked photoresist layer. Sensing region may include a sharp tip of at least one electrode; therefore, just individual sensing regions for capturing and attachment of a single cell may be opened. Cells may be dropped on the biosensor without the assistance of any instrument such as a micropipette or microfluidic systems and consequently, single cells may be captured or trapped within the sensing regions for further analysis. In an exemplary embodiment, a noticeable population of the cells may be dropped to be seeded on the surface of the biosensor and single cells may be located in a desired location including a sensing region around sharp tip of an electrode.

In some exemplary implementations, a single cell's resistance to anticancer drugs may be analyzed by bioelectrical monitoring utilizing the disclosed biosensor. Accordingly, in some exemplary embodiments, the exemplary biosensor disclose herein may be developed for noninvasive monitoring of the effect of anticancer drugs on single cancer cells based on nano-roughened substrate equipped with an array of electrodes.

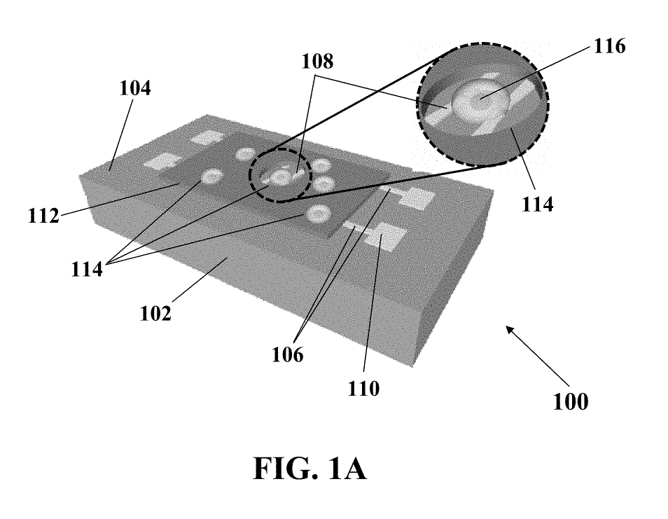

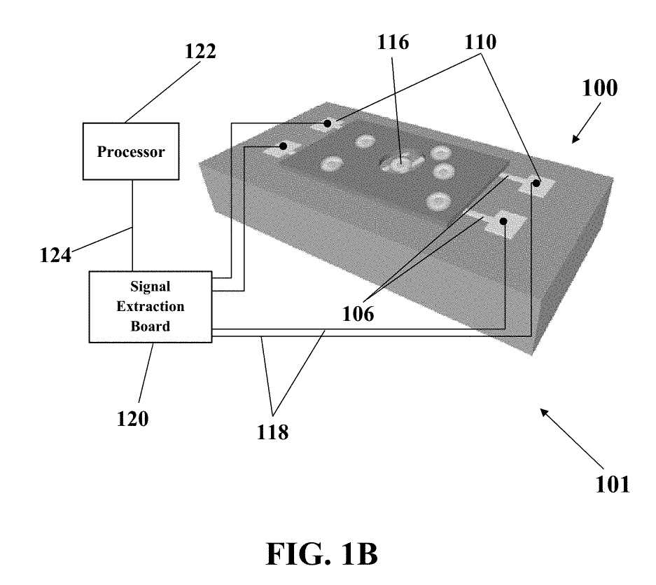

FIG. 1A shows a schematic view of a biosensor 100 for single cell analysis, consistent with one or more exemplary embodiments of the present disclosure. The biosensor 100 may include a substrate 102, where the substrate 102 may include a roughened surface 104, an array of electrodes 106 patterned on the roughened surface 104, where each electrode of the array of electrodes 106 may include a distal tip 108 and a proximal end 110, and a passivation layer 112. The passivation layer 112 may deposit or cover on top of the biosensor 100. The passivation layer 112 may include a microwell 114 around the distal tip 108 of an electrode, where a single cell 116 may be trapped within the microwell 114 and adhered onto the distal tip 108 of the electrode.

As used herein, a “passivation layer” may refer to an electrically insulator layer that may include a photoresist layer. The passivation layer may be configured to electrically isolate the biosensor surface and the array of electrodes 106 except the distal tip 108 and the proximal end 110 of each electrode that may result in more accurate electrical signals received from the array of electrodes 106.

In some exemplary implementations, a single cell 116 may be trapped within the microwell 114 through a simple procedure. In an exemplary embodiment, the procedure may include dropwise inserting a solution of a population of cells, where the solution may include the single cell 116 and the single cell 116 may be captured and trapped within the microwell 114. That it, the solution may be deposited as a series of drops, and a single cell is captured from the solution within microwell 114.

In some exemplary implementations, the substrate 102 may comprises of a quartz substrate, a glass substrate, a silicone-based substrate, a Pyrex substrate, a Poly(methyl methacrylate) (PMMA) substrate, or combinations thereof. In an exemplary embodiment, the substrate 102 may include a substrate with a thickness between about 0.5 mm and about 3 mm, for example about 1 mm.

In some exemplary implementations, the roughened surface 104 may comprise a nano-roughened surface that may include nanosized features with diameter of less than about 100 nm and height of less than about 200 nm, for example, between about 100 nm and about 200 nm. In an exemplary embodiment, a nano-roughened quartz substrate may be used instead of using a conventional ligand-mediated cell adhesion to the substrate, in order to increase the cell adhesion onto the substrate and also to enhance the resolution of signal extraction from the trapped singe cell for further analysis, for example, for detecting the trace of drug treatment in much lower doses.

In exemplary implementations, the array of electrodes 106 may include an array of gold (Au) microelectrodes, which may be patterned and deposited on the roughened surface 104. In an exemplary embodiment, the array of electrodes 106 may have a thickness of less than about 20 nm, for example, a thickness between about 10 nm and about 15 nm. In an exemplary embodiment, the array of electrodes 106 may include a Titanium/Gold (Ti/Au) bilayer with a thickness of less than about 20 nm. In different implementations, each electrode of the array of electrodes 106 may include an electrical path with a distal tip 108 and a proximal end 110. The distal tip 108 may include a sharp tip of each electrode that may provide a direct contact to the single cell 116 that may be trapped in the microwell 114 and adhered onto the distal tip 108. Hence, an accurate electrical measurement of the cell's behavior may be achieved through this direct and close contact between the single cell 116 and the distal tip 108 of at least one electrode of the array of electrodes 106.

In some implementations, the biosensor 100 may further include a layer of Titanium (Ti) between the array of electrodes 106 and the roughened surface 104. The Ti layer may be configured to improve the adhesion of the array of gold electrodes 106 onto the roughened surface 104.

In some implementations, the passivation layer 112 may include an electrically insulated layer that may be formed by spinning a photoresist layer on an area on top of the biosensor 100 except the distal tip 108 and the proximal end 110 of the array of electrodes 106. The distal tip 108 may be preserved in a non-passivated state in order to provide a sensing region within the microwell 114 for capturing and adhering the single cell 116 on at least one distal tip 108. In addition, the proximal end 110 of the array of electrodes 106 may be preserved in a non-passivated state in order to provide an electrical connection for electrical signal extraction from the single cell 116. In an exemplary embodiment, the passivation layer 112 may include an overbaked photoresist polymeric layer, for example, a photoresist polymeric layer with a thickness of less than about 5 μm.

The passivation layer 112 may include the microwell 114, which may provide an open access to the distal tip 108 of an electrode of the array of electrodes 106 for capturing and adhering a single cell onto the distal tip 108. Hence, the distal tip 108 of the electrodes within the microwell 114 may be exposed to a cell-contained solution and other regions of the surface of the biosensor 100 may be passivated by the passivation layer 112. Accordingly, the microwell 114 may surround at least one distal tip 108 and may prepare a suitable space for preferential adhesion of cells. In an exemplary embodiment, the microwell 114 may include a circular cavity with a diameter between about 20 μm and about 50 μm and a depth between about 2 μm and about 5 μm.

In some implementations, the biosensor 100 may be placed within an electrical measurement system for single cell analysis. A schematic view of an implementation of an electrical measurement system is shown in FIG. 1B as the electrical measurement system 101. The electrical measurement system 101 may include the biosensor 100, an electrical signal extraction board 120, and a processor 122. The electrical signal extraction board 120 may be configured to apply a voltage on the array of electrodes 106 and to receive an electrical signal from the array of electrodes 106. Furthermore, the processor 122 may be configured to store and analyze the electrical signal received by the electrical signal extraction board 120. The proximal ends 110 of the array of electrodes 106 of the biosensor 100 may be electrically connected to the electrical signal extraction board 120, for example, via electrical paths 118. In addition, the electrical signal extraction board 120 may be electrically connected to the processor 122, for example, via electrical paths 124, in order to send the electrical signal received from a single cell 116. In an exemplary embodiment, the electrical signal may include an electrical resistance or an electrical impedance of the single cell 116. In another exemplary embodiment, the electrical signal may include an electrical impedance of the membrane of single cell 116. In an exemplary embodiment, the applied voltage may be about 40 mV and the voltage may be applied with a frequency between about 1 KHz and about 150 KHz.

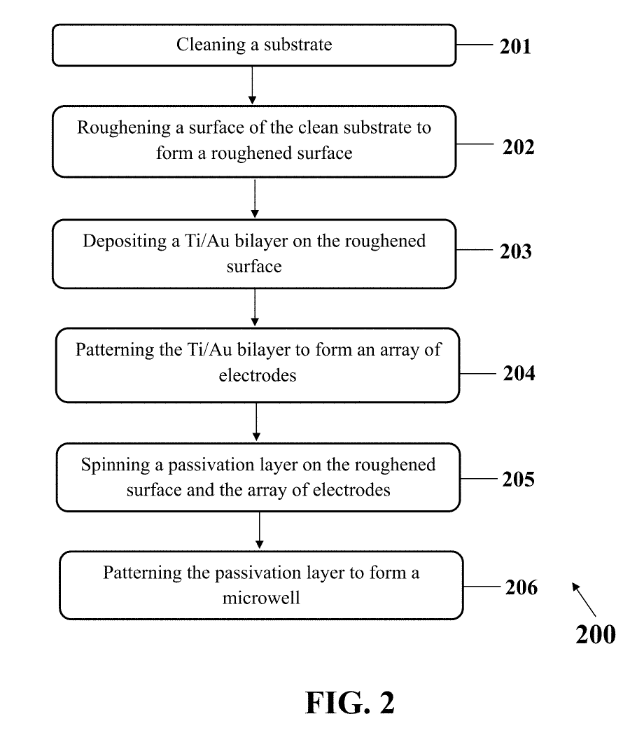

In an exemplary embodiment of the present disclosure, a method for fabricating a biosensor for single cell analysis is disclosed, such as exemplary biosensor 100 (FIG. 1A). FIG. 2 shows method 200 for fabricating a biosensor, for example, the biosensor 100 (FIG. 1A) for single cell analysis, consistent with one or more exemplary embodiments of the present disclosure.

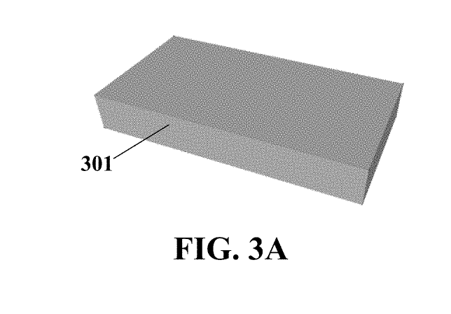

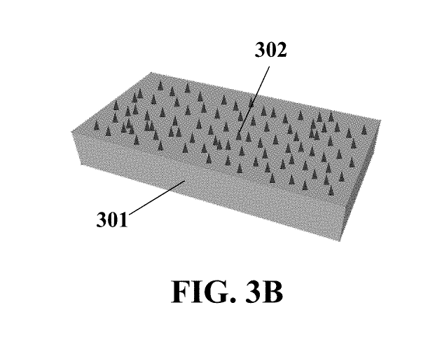

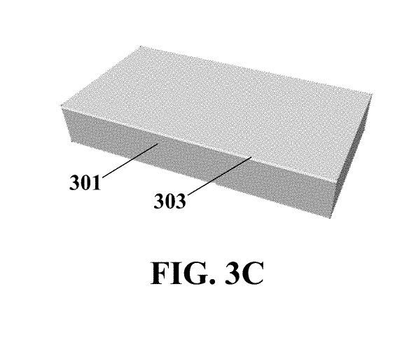

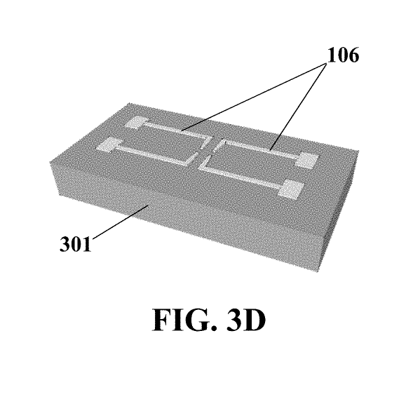

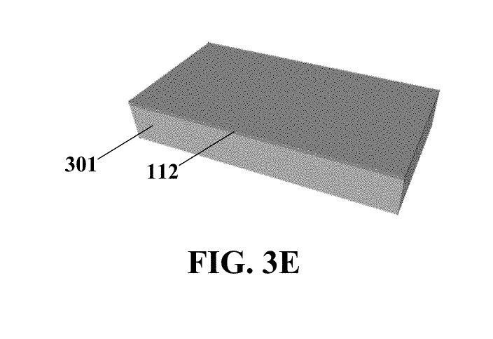

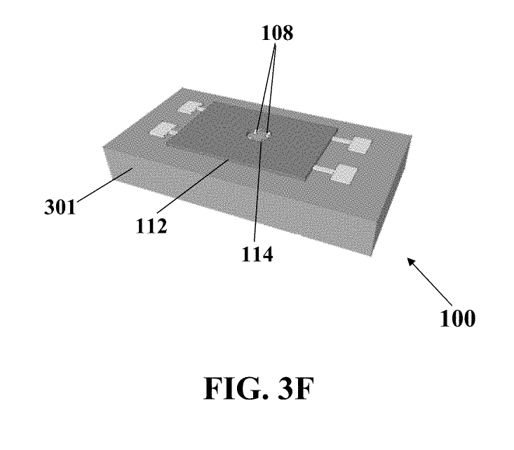

Referring to FIG. 2, the method 200 may include cleaning a substrate (step 201), roughening a surface of the clean substrate to form a roughened surface on the clean substrate (step 202), depositing a Titanium/Gold (Ti/Au) bilayer on the roughened surface (step 203), patterning the Ti/Au bilayer to form an array of electrodes (step 204), spinning a passivation layer on the roughened surface and the array of electrodes (step 205), and patterning the passivation layer to form a microwell (step 206). In addition, for purposes of clarity to the reader, FIGS. 3A-3F show implementations of the steps of method 200.

In step 201, the substrate may be cleaned in order to remove trace amounts of organic residues from the substrate, therefore a clean substrate may be obtained. One implementation of the clean substrate is shown in FIG. 3A represented by the clean substrate 301. In an exemplary embodiment, the substrate may be cleaned using a piranha solution, which may include a mixture of concentrated sulfuric acid and hydrogen peroxide with a ratio of about 3:1.

In step 202, a surface of the clean substrate may be roughened, in order to form a roughened surface on the clean substrate. FIG. 3B shows implementation of the roughened surface 302 formed on a top surface of the clean substrate 301. In an exemplary embodiment, roughening the surface of the clean substrate may include placing the clean substrate 301 in a buffer solution for less than 30 min. For example, the clean substrate may be placed in a buffer HF solution for about 15-20 min to create the roughened surface 302. In another exemplary embodiment, roughening the surface of the clean substrate may include holding the clean substrate 301 in a reactive ion etching (RIE) system. In an exemplary embodiment, the roughened surface 302 may include a nano-roughened surface that may include nano features with diameter of less than about 100 nm and height of less than about 200 nm.

In step 203 with a reference to FIG. 3C, a Titanium/Gold (Ti/Au) bilayer 303 may be deposited on the roughened surface and the Ti/Au bilayer 303 may include a Ti layer deposited on the roughened surface and an Au layer deposited on the Ti layer. In an exemplary embodiment, the Ti/Au bilayer 303 may be deposited on the roughened surface using a sputtering process, for example, using a RF-sputtering system. In an exemplary embodiment, the Ti layer may include a layer with a thickness of less than about 5 nm and the Ti layer may be configured to improve the Au layer adhesion onto the roughened surface. The Au layer may include a layer with a thickness of less than about 20 nm.

In step 204 with a reference to FIG. 3D, the Ti/Au bilayer 303 may be patterned, in order to form the array of electrodes 106 on the roughened surface 302. In an exemplary embodiment, the Ti/Au bilayer 303 may be patterned using a lithography process to form the architecture of the array of electrodes 106 of the biosensor 100.

In step 205 with a reference to FIG. 3E, a passivation layer 112 may be spun on the roughened surface 302 and the array of electrodes 106 on the surface of the substrate 301. In an exemplary embodiment, spinning the passivation layer 112 may include coating or depositing a photoresist layer on the roughened surface 302 and the array of electrodes 106, so that the passivation layer 112 may totally covered the surface of the substrate 301. In an exemplary embodiment, the thickness of passivation layer 112 may be less than about 5 μm.

In step 206 with a reference to FIG. 3F, the passivation layer 112 may be patterned to form a microwell 114, wherein the microwell 114 may include a non-passivated cavity around a distal tip 108 of one or more electrodes of the array of electrodes and the microwell 114 may form a trap for capturing a single cell and adhering the single cell on the distal tip 108 of the electrode. In an exemplary embodiment, the microwell 114 may include a non-passivated circular region around at least one electrode tip of the array of electrodes 106 that may be formed for a single cell attachment. The microwell 114 may enhance the probability of only a single cell attachment onto an electrode distal tip.

In an exemplary implementation, the method 200 may further include overbaking the biosensor 100 fabricated using exemplary method 200 at a temperature of more than about 100° C., in order to remove toxic materials from the passivation layer 112. In an exemplary embodiment, the fabricated biosensor 100 may be overbaked at a temperature of more than about 100° C. for about 30 min to about 2 hours, in order to remove the toxic materials from the passivation layer 112.

### EXAMPLE 1

### Fabrication of Biosensor

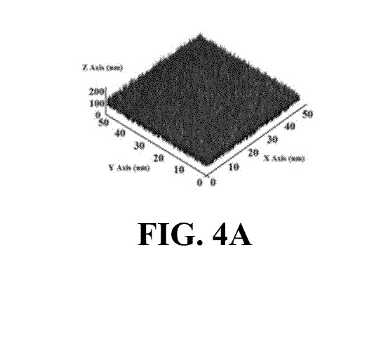

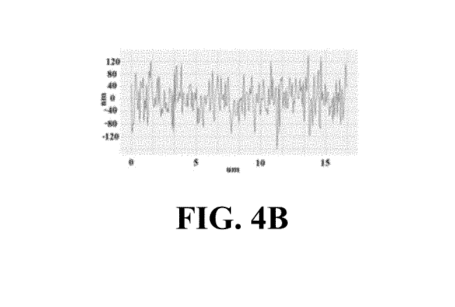

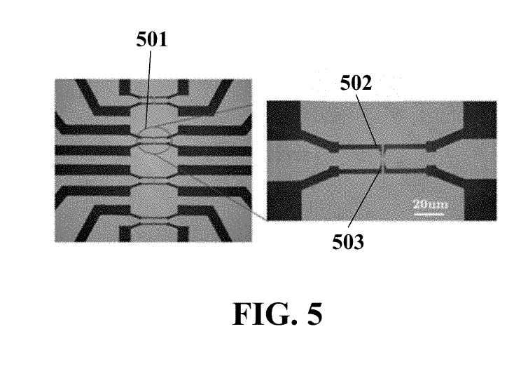

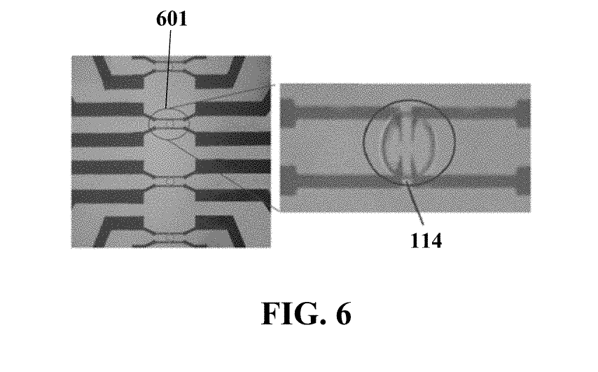

In this example, a quartz sample as a substrate was cleaned using a piranha solution in order to remove trace amounts of organic residues from the substrate. The cleaned substrate was then placed in a buffer HF solution (about 10%) for about 15-20 min to create a nano-roughened surface. FIGS. 4A and 4B show respective 3D (FIG. 4A) and line (FIG. 4B) atomic-force microscopy (AFM) image of the obtained nano-roughened surface. It may be observed from these figures that nano-roughening process induced nano features with diameter of less than about 100 nm and height of about 100-200 nm. Subsequently, a thin layer of titanium with a thickness of about 1-3 nm was deposited on the nano-roughened surface and after that a layer of gold with a thickness of about 10-15 nm was coated on the titanium layer using a RF-sputtering system. The Ti/Au bilayer was patterned by standard lithography process to form the biosensor electrodes. FIG. 5 shows an optical image of the patterned array of electrodes (left) and an optical image of a magnified portion 501 of the patterned array of electrodes (right) representing a distance of about 7 μm between two adjacent electrodes 502 and 503. The samples were then passivated using a photoresist layer in such a way that a non-passivated circular region (a microwell) around the electrode tips was formed for cell attachment. The thickness of passivation layer was about 2 μm and the microwell which was formed around the electrodes tips may enhance the probability of only a single cell attachment. Then, the fabricated biosensor was overbaked at a temperature of about 120° C. for about 45 min to remove the toxic materials from the photoresist layer. FIG. 6 shows an optical image of the array of electrodes with patterned passivation layer including microwells (left) and an optical image of a magnified portion 601 of the array of electrodes with patterned passivation layer (right) before cell attachment. The microwell 114 as a sensing region for adhering or attaching a single cell onto the electrodes may be distinguished from this figure.

### EXAMPLE 2

### Cell Culture

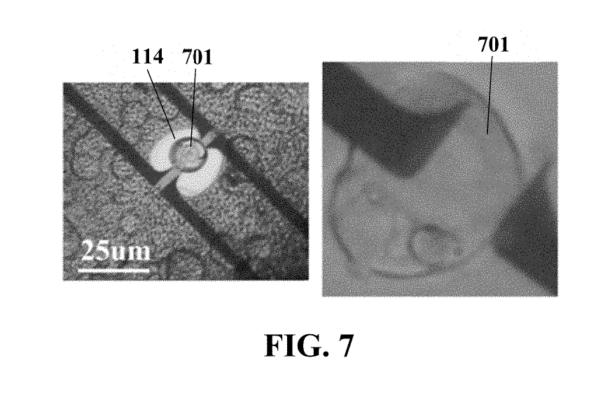

MCF-7 cell lines which were acquired to be used in this example. The cell lines have been separated from grad I human breast tumors. The cell lines were maintained at 37° C. in an incubator (about 5% CO₂, about 95% clean air) and in RPMI-1640 medium supplemented with about 5% fetal bovine serum and about 1% penicillin/streptomycin. The fresh medium was replaced every other day. In order to detach cell from the substrate and become suspended in cell solution, cells was trypsynized. As performing cell culturing process at room temperature of about 20° C. -22° C., trypsinization should be done in less than about 6 minutes to minimize the influence of trypsinization. The cell solution was dropped onto an exemplary biosensor, which was fabricated according to EXAMPLE 1, and then the biosensor was maintained in an incubator for about 4 hours to reach cells attachment on the biosensor surface and trapping single cells within microwells of the biosensor. FIG. 7 shows an optical image of an individual MCF-7 single cell 701 trapped within the microwell 114 and attached on the electrodes tip after dropping the cells solution (time=0) (left) and an optical image of a magnified portion around the MCF-7 single cell 701 after cell attachment (time=2 hours) (right).

### EXAMPLE 3

### Biocompatibility of the Biosensor

In this example, interaction of living cells by cured overbaked photoresistant passivation layer of an exemplary fabricated biosensor may reveal the biocompatibility of the biosensor with a passivation layer with living cells as experimented here by a 3-(4,5-dimethylthiazol-2-yl)-2,5-diphenyltetra-zolium bromide (MTT) assay and by optical imaging from morphology and growth of the cells on the biosensor.

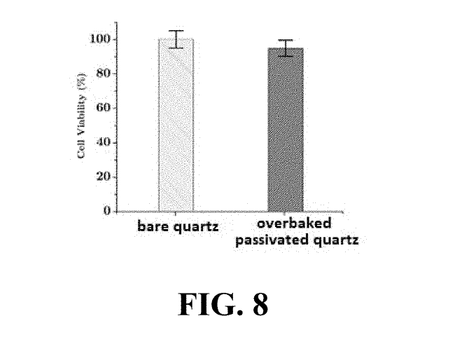

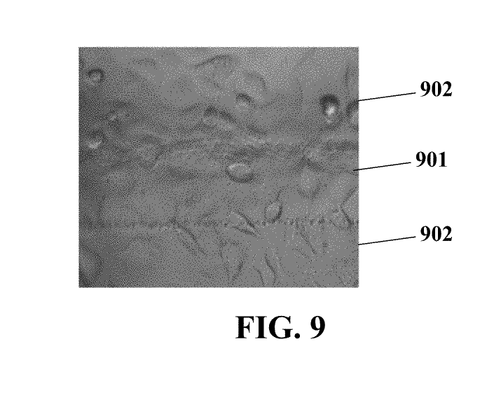

FIG. 8 shows results of a MTT assay for MCF-7 cells cultured on a bare quartz and a quartz coated by an over-baked passivation layer, where a good biocompatibility may be observable for the biosensor with a passivation layer. Furthermore, FIG. 9 illustrates optical microscopy images from a quartz surface patterned by a passivation layer and overbaked at about 120° C. for about 45 min. The extension and geometry of the attached cells may be similar for bare region 901 and passivated region 902 of the quartz surface.

### EXAMPLE 4

### Extreme Drug Resistance (EDR) Single Cell Analysis

In this example, MCF-7 cancer cells may be time tracked under the effect of Paclitaxel (PTX) and Mebendazole (MBZ) anti-tubulin drugs with low and high doses. Inducing non-regulated depolymerization and polymerization in tubulin structures of the single cancer cells may be monitored by the electrical signals recorded before and after drug treatment. Electrical responses of single cells to their incubation with drugs may reflect their vitality and biological states which may be confirmed by another cell analysis technique such as confocal imaging. The seeded MCF-7 cancer cells were exposed to known concentrations of anticancer drugs which induced polymerization (PTX) and depolymerization (MBZ) in tubulin structure of the cancer cells. The electrical signals were recorded before and after drug treatment up to about 48 hours. Also, optical and confocal imaging were investigated to highlight the biological translation of electrical pattern achieved from cancer cells.

Exemplary biosensors with cell solutions dropped onto them (as described herein in EXAMPLE 2) were maintained in an incubator for about 4 hours to reach cells attachment on biosensors surface. MBZ and PTX antitubulin drugs were added into the microwells containing single cells in low concentrations (about 2.1 nM for MBZ and about 0.1 nM for PTX) and high concentrations (about 10.5 nM for MBZ and about 1 nM for PTX) concentrations. The signals were recorded 2 hours, 5 hours, 8 hours, 20 hours, 32 hours, and 44 hours after drug addition that means 6 hours, 9 hours, 12 hours, 24 hours, 32 hours, and 44 hours after the initial drop. Electrical signals Measurements were collected in frequencies ranged from about 1 KHz to about 60 KHz by impedance measurement system (electrical signal extraction board) at about every 2 hours. A small “ac” sinusoidal signal was applied to the sensors. For different frequencies of the “ac” signal, the sensitivity of the sensor would vary which is called as the frequency characteristics. The bias voltage of the system was about 40 mV which wasn't destructive for cellular metabolism and a series resistance was used as current limiter element. For each cell trapped in each microwell of the biosensors, four electrodes may be used for signal extraction in two states (with up-down or right-left neighbor electrodes). The final result may be the average of the several signals extracted from the different sensors on a chip (several microwells on a biosensor) to consider cellular heterogeneity and different positioning of the cells relative to the electrodes and other parameters.

For confocal imaging, confocal images were taken from MBZ and PTX treated cells in high and low doses. Non treated cells were imaged as control (CTRL) for comparison. Accordingly, biosensors were washed with PBS and permeablized with microtubule stabilizing buffer [80 mM PIPES-KOH (pH 6.8), 5 mM EGTA, and 1 mM MgCl₂ containing 0.5% Triton X-100] for about 5 min at room temperature before being fixed with chilled absolute methanol for about 10 min at about −20 ° C. Fixed trapped single cells were washed and incubated with monoclonal mouse anti-α-tubulin antibody for about 1 hour at room temperature followed by incubation with FITC-conjugated antimouse IgG antibody.

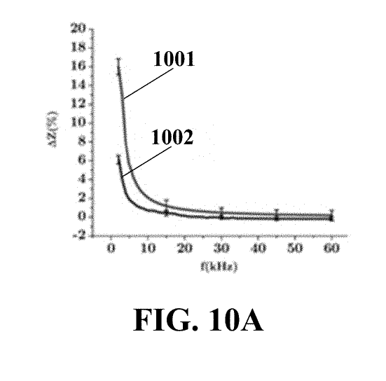

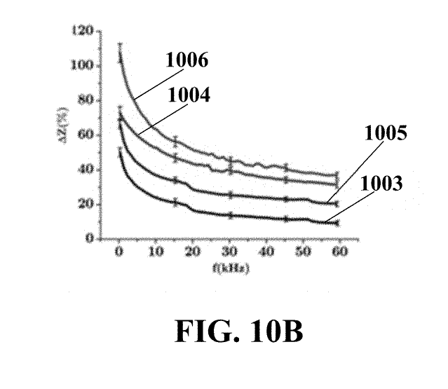

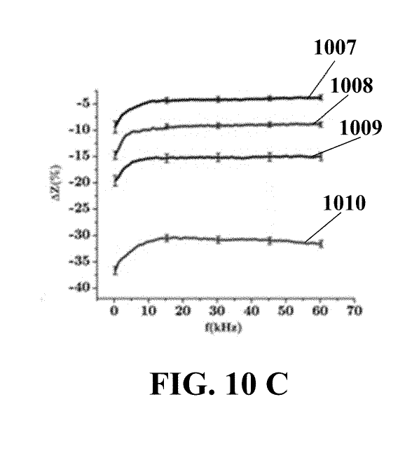

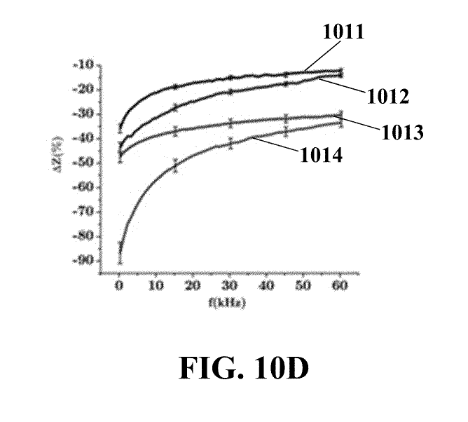

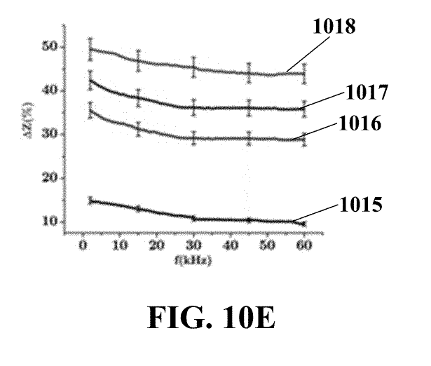

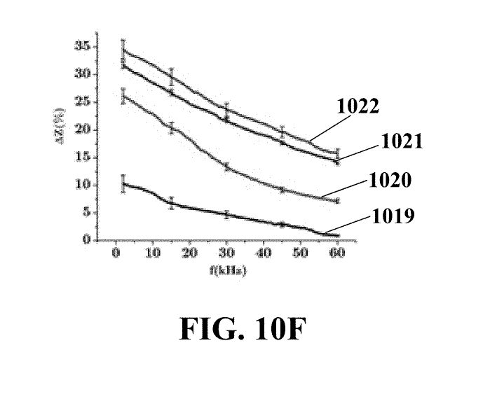

FIGS. 10A-10F show impedance diagrams for a MCF-7 single cell seeded and trapped in a microwell between electrodes about 4 hours after dropping the solution containing cells, which was assumed as an initial time (T=0) for all measurement experiments. After the attachment of the cell on the region between recording or sensing electrodes (after about 4 hours), observable increment in impedance was recorded with respect to media solution covered state (curve 1001 Vs. curve 1002 in FIG. 10A). T parameter was defined as a time merit started after dropping of the cells (t=4 hours was assumed equal to T=0). Delta Z (ΔZ) may include the difference between impedance at about 4 hours after cell seeding (equal to T=0) and the 4+x^{th} hours (x may be equal to 2, 5, 8, etc.), divided by the impedance at T=0. Average impedance of all frequencies were used for comparison. For better comprehension of impedimetric regime, the impedance changes of the CTRL cell were plotted in subsequently next time periods (about 6^{th}, 9^{th}, 12^{th} and 24^{th} hours) with respect to the impedance of the sensor in 4^{th} hours after dropping the cells solution that are shown by curves 1003 (at T=2 or t=6 hours), 1004 (at T=5 or t=9 hours), 1005 (at T=8 or t=12 hours), and 1006 (at T=20 or t=24 hours) in FIG. 10B. It may be observable that the impedance of the biosensor exhibited about 17%, 40%, 30% and 50% increase in respective 2^{nd}, 5^{th}, 8^{th} and 20^{th} hours after the first signal extraction from the seeded single cell, respectively. As the first signaling was recorded about 4 hours after dropping the cells contained solution, the reference point of recording time was changed from 0^{th} hour to 4^{th} hours. So each interval of time that may be represented by T may mean the time passed from the 4^{th} hours after dropping the cells on the surface of the biosensor.

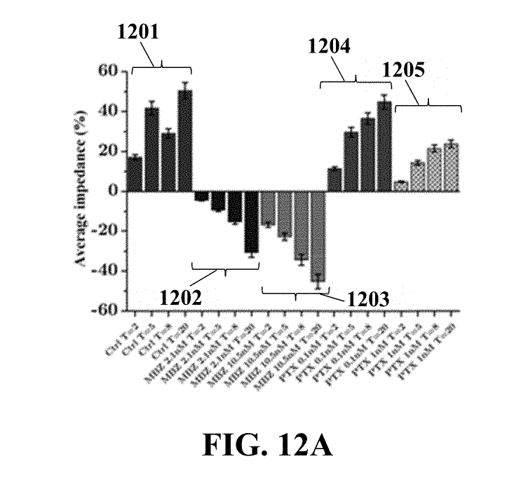

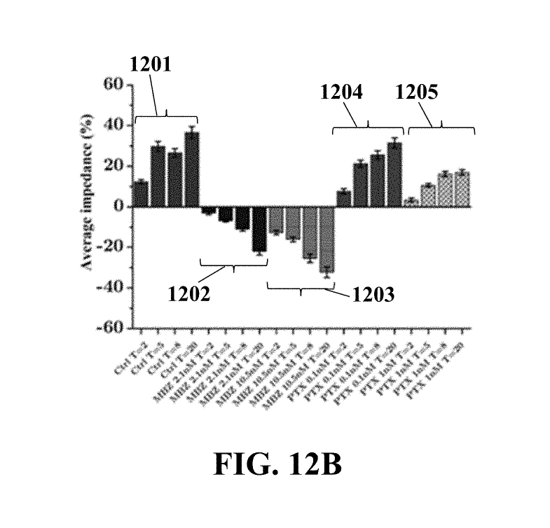

Referring again to FIG. 10B, the rate of impedance enhancement was ascending from T=2 to T=5. After that, the cell exhibited a reduction in the rate of impedance increment (from T=5 to T=8). When cancer cells enter into spreading stage that may be about 7 hours after their attachment on the surface, they exhibited reduced impedance response. The reason of such reduction may be related to the changed ratio of membranes fatty acids and phospholipid structures during cancer transformation. This may result in weaker mechanical and dielectric functionality of cancer cell membrane transformations and when cancer cells enter to a spreading stage, their ability to block the incoming current becomes weaker than normal cells which could be characterized as beta dispersion behavior of the cells. In the first steps of the spreading (T=5), cell covers more area of the sensing electrode and cause an increase of the impedance. But as the cancer cell stretched more, membrane's current blocking ability decreased due to thinner structure interfaced with the electrodes and current may be passed through the membrane. However the electrode structure and precision, for example, by using silicon nanowires or using a nano-roughened surface may be very important for detection of these fine signals (see FIGS. 12A and 12B described herein below). This result may be in a well match with a comparison between curves 1004 and 1005. From T=8 (curve 1005) to T=20 (curve 1006), the cell continued its impedance increment. Such increase may be in a well corroboration with other reports on enhancement of the cells impedance during growth and mitosis. This would be a good calibration for monitoring the impedance response of single cell for further treatments, for example, with anticancer or antitubulin drugs.

It should be noted that the exemplary sensing procedure was straightforward and was predominated by dielectric and beta dispersion changes due to the direct attachment and stretch of cells on the electrodes of the biosensor. Beta dispersion as one of the intrinsic bioelectrical properties of the cell membrane in response to AC electrical excitation may be one of the key parameters involved in the present diagnosis/analysis approach. This phenomenon may be related to the ability of a biological cell membrane in filtering out low frequency currents and allowing high frequency ones to pass through. As the cancerous cells may have less ability in blocking the current flow into their membrane, the beta dispersion effects are different from healthy cells.

Referring to FIGS. 10C and 10D, electrical responses of the individual microwells of separate two exemplary biosensors, which were covered by MCF-7 single cells, treated by two different concentrations of MBZ, including about 2.1 nM (FIG. 10C) and about 10.5 nM (FIG. 10D) for about 20 hours (between 4^{th} to 24^{th} hours after dropping the cells on the surface). These figures represent a dose dependent decrease in impedance of the cells in time evolution. The associated curves are designated by curves 1007 (at T=2), 1008 (at T=5), 1009 (at T=8), and 1010 (at T=20) for those single cells treated by about 2.1 nM MBZ (FIG. 10C) and curves 1011 (at T=2), 1012 (at T=5), 1013 (at T=8), and 1014 (at T=20) for those single cells treated by about 10.5 nM MBZ (FIG. 10D). Such reduction for 2.1 nM treated cell was about 5%, 10%, 15% and 30% at T=2 hours, 6 hours, 8 hours, and 20 hours, respectively (FIG. 10C). Also, impedance reduction was about 15%, 25%, 35% and 45% in the similar time intervals after treating the cell covered electrodes by about 10.5 nM of MBZ (FIG. 10D). Inducing apoptosis by treatment of MBZ treatment would be one of the major reasons causing such reduced impedance. It may be known that apoptotic cells exhibited serious reduction in their impedance. However, the rate of impedance reduction versus sweeping frequency was sharper at T=20 (curves 1010 and 1014) meanwhile it was so slower in T=2 (curves 1007 and 1011) or

T=5 (curves 1008 and 1012). Time progression after treating the cells by the drug might sharply present the drug effect on the dielectric parameter of the cells as a frequency dependent response.

Referring to FIGS. 10E and 10F, incubating the cell by PTX drug, as a MT (microtubule) polymerizing agent, resulted in different electrical behavior from those of resulted by MBZ. The associated curves to PTX treated cells are designated by curves 1015 (at T=2), 1016 (at T=5), 1017 (at T=8), and 1018 (at T=20) for those single cells treated by about 0.1 nM PTX (FIG. 10E) and curves 1019 (at T=2), 1020 (at T=5), 1021 (at T=8), and 1022 (at T=20) for those single cells treated by about 1 nM PTX (FIG. 10F). Time dependent increase in the impedance of PTX treated single cell was observed with slower rate and values than that of CTRL (control) (FIG. 10B). The impedance increment in 0.1 nM PTX treated cells was about 10%, 30%, 35%, and 45% at T=about 2 hours, 5 hours, 8 hours, and 20 hours, respectively. Such increment in high dose PTX (1 nM) treated sample was about 5%, 15%, 20%, and 25% in similar order of time intervals. Enhanced density of the MTs, as dielectric macromolecules in the cytoplasm of the cell because of polymerization might block the current through PTX treated cells and increase the impedance responses of the cells. Moreover, the impedance increment because of overpolymerized MTs (after PTX treatment) might compensate the impedance reduction resulted by spreading stage. Finally, a monotonously increasing regime was observed in PTX treated cells with both concentrations, but in different values. Sharp reduction in time evaluated impedance of MBZ cells and in contrast moderate increment in time evaluated electrical response of PTX treated single cell (in comparison with CTRL sample) might be correlated with pathological effects of Taxol based drugs on cellular metabolism, because such drugs wouldn't induce noticeable disruption on cellular function before their entrance into mitotic state. As a result, these drugs wouldn't induce serious impedance disruption on the cell in pre-mitotic cycles (from T=2 to T=20). Due to averaging of the five different experiments (CTRL cell, treated cells with low and high concentrations of MBZ, and treated cells with low and high concentrations of PTX), it could be supposed that the morphology and geometry of the cells haven't effect on a cell electrical response. This would corroborate the similar electrical responses extracted from the individual device at T=0.

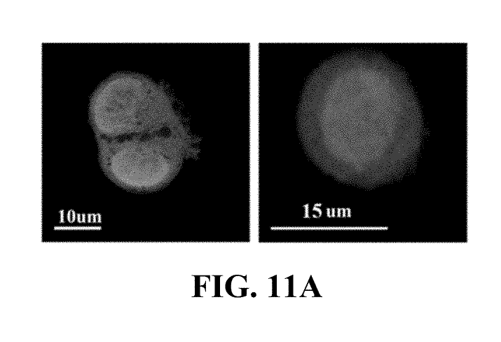

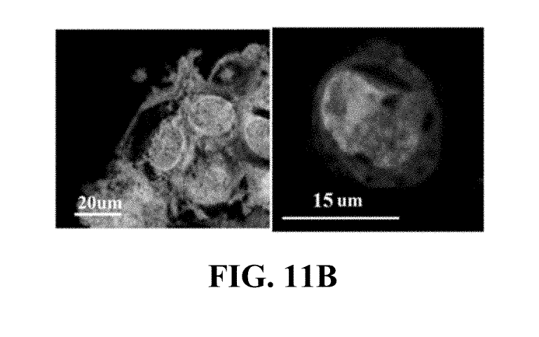

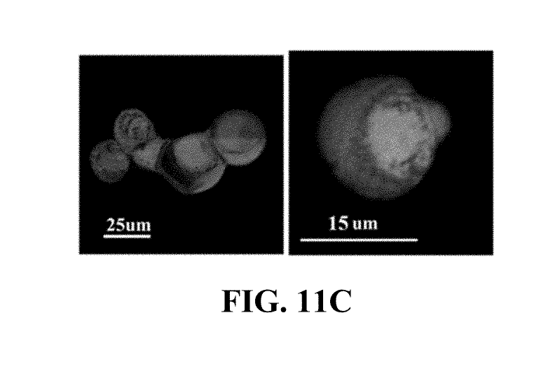

FIGS. 11A-11C show confocal images taken from CTRL (FIG. 11A), 10.5 nM MBZ treated (FIG. 11B) and 1 nM PTX treated (FIG. 11C) cells at about 20 hours after drug treatment with two magnifications represented in left and right of these figures. These figures may reveal different effects of the drug on cytoskeletal structure of the cells. CTRL cells exhibited normal mitosis and division of nucleus and cytoskeleton structure with conformal shape and same ratio between new formed cells. No entrance to even primary cycles of mitosis, degraded distribution of cytoskeleton and monoastered microtubules are observable indications of MBZ treated cells in comparison with CTRL sample. In addition, images of the PTX treated cell indicated the aggregated MTs as polymerizing effect of Taxol on cytoskeletal structure which induced mitotic arrest in the cell and deformed the nucleus during the mitosis. Such biological changes may better indicate the considerable different electrical responses between CTRL and drug treated cells attached on the electrodes at T=20 hours. Perturbed cytoskeletal structure would disturb the cellular functions by activation of some pathways such as caspase 3 or 9 and finally the cells might be arrested in mitotic cycle or entered to apoptosis before any growth.

FIGS. 12A and 12B show mean impedance changes of the single cells treated by various doses of the drugs in a comparative style for a single cell on a biosensor with a nano-roughened surface (FIG. 12A) and on a biosensor with anon-nano-roughened (smooth) surface (FIG. 12B). Time dependent behavior of non-treated control cells (columns 1201) showed continues increment in impedance (except in spreading stage). Treating the cells with MBZ induced continues reduction in the impedance which exhibited a linier correlation with doses of the drug (columns 1202 for 2.1 nM MBZ treatment, and columns 1203 for 10.5 nM MBZ treatment). By this kind of representation, just a quick look is enough to indicate the reductive effect of MBZ on cells impedance. PTX treated cells showed moderate impedance increment with dose dependent reductive regime (columns 1204 for 0.1 nM PTX treatment, and columns 1205 for 1 nM PTX treatment). This would corroborate non apoptotic effect of PTX on MCF-7 cells. To elaborate the effect of nano-roughening on the precision of the measurement and the biosensor sensitivity, the average impedance of the nano-roughened biosensor measured at the frequency of about 4 kHz (FIG. 12A) was compared by that in a biosensor with smooth surface (FIG. 12B). The impedances in all of swap frequencies were calculated and the mean value was extracted by assigning the sharper difference in impedance (in lower frequency) in similar weight to slower differences (in higher frequencies). In addition, at least five experiments were made to ensure about impact of nano-roughening. As shown in this FIG. 12B, the percent of the impedance changes are about 20% greater in a nano-roughened biosensor. So, nanoscale geometry of the electrodes may play a crucial role in a detection mechanism and sensor precision.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1- A biosensor for single cell analysis, comprising:
a substrate with a roughened surface;
an array of electrodes patterned on the roughened surface, each electrode comprising a distal tip and a proximal end; and
a passivation layer deposited on top of the substrate and the array of electrodes, wherein: the passivation layer forms a microwell around the distal tip of anelectrode of the array of electrodes, the mircowell is configured to trap a single cell, and the single cell is adhered onto the distal tip of the electrode.

2- The biosensor of claim 1, wherein the single cell is trapped within the microwell by drop-by-drop insertion of a solution of a population of cells, the solution comprising the single cell.

3- The biosensor of claim 1, wherein the roughened surface comprises a nano-roughened surface including nanosized features with diameter of less than 100 nm and height of less than 200 nm.

4- The biosensor of claim 1, wherein the substrate comprises one of a quartz substrate, a glass substrate, a silicone-based substrate, a Pyrex substrate, a Poly(methyl methacrylate) (PMMA) substrate, or combinations thereof

5- The biosensor of claim 1, wherein the microwell comprises a circular cavity with a diameter between 20 μm and 50 μm and a depth between 2 μm and 5 μm.

6- The biosensor of claim 1, wherein the array of electrodes comprises an array of Gold (Au) microelectrodes with a thickness of less than 20 nm.

7- The biosensor of claim 1, wherein the array of electrodes comprises a Titanium/Gold (Ti/Au) bilayer with a thickness of less than 20 nm. 8- The biosensor of claim 1, wherein the biosensor further comprises:
a layer of Titanium (Ti) between the array of electrodes and the roughened surface, configured to improve the adhesion of the array of electrodes onto the roughened surface.

9- The biosensor of claim 1, wherein the passivation layer comprises an electrically insulator layer and is formed by spinning a photoresist layer on an area on top of the substrate and array of electrodes except the distal tip and the proximal end of the array of electrodes.

10- The biosensor of claim 1, wherein the passivation layer comprises an overbaked photoresist polymeric layer with a thickness of less than 5 μm.

11- A method for fabricating a biosensor for single cell analysis, comprising:
cleaning a substrate;
roughening a surface of the clean substrate to form a roughened surface on the clean substrate;
depositing a Titanium/Gold (Ti/Au) bilayer on the roughened surface, wherein the Ti/Au bilayer comprises a Ti layer deposited on the roughened surface and an Au layer deposited on the Ti layer;
patterning the Ti/Au bilayer to form an array of electrodes;
spinning a passivation layer on the roughened surface and the array of electrodes; and
patterning the passivation layer to form a microwell, wherein the microwell comprises a cavity around a distal tip of an electrode of the array of electrodes and the microwell forms a trap for capturing a single cell and adhering the single cell on the distal tip of the electrode.

12- The method according to claim 11, wherein roughening the surface of the clean substrate comprises holding the clean substrate in a reactive ion etching (RIE) system.

13- The method according to claim 11, wherein roughening the surface of the clean substrate comprises placing the clean substrate in a buffer HF (Hydrogen fluoride) solution for less than 30 min.

14- The method according to claim 11, wherein the bilayer of Ti/Au is deposited on the roughened surface using a sputtering process.

15- The method according to claim 11, wherein patterning the Ti/Au bilayer comprises patterning the Ti/Au bilayer using a lithography process.

16- The method according to claim 11, wherein spinning a passivation layer comprises depositing a photoresist layer on the roughened surface and the array of electrodes.

17- The method according to claim 11, wherein patterning the passivation layer comprises patterning the passivation layer using a lithography process.

18- The method according to claim 11, wherein the method further comprises:
overbaking the biosensor at a temperature of more than 100° C. to remove toxic materials from the passivation layer.
